(19) United States
(12) Patent Application Publication
(10) Pub. No.: US 2004/0171538 A1
(43) Pub. Date: Sep. 2, 2004

(54) Anticoagulants and their uses

(76) Inventors: Nils Von Sicard, Swansea (GB); Michael Morris, Indiana, IN (US); Asgar Electricwala, Salisbury (GB); Roger Munro, Swansea (GB)

Correspondence Address: KLARQUIST SPARKMAN, LLP, 121 SW SALMON STREET, SUITE 1600, PORTLAND, OR, 97204, US

(21) Appl. No.: 10/470,249

(22) PCT Filed: Jan. 21, 2002
(86) PCT No.: PCT/GB02/00251

(30) Foreign Application Priority Data
Jan. 24, 2001 (GB) 0101879.5

(51) Int. Cl. A61K 38/00; C07H 21/04

(52) U.S. Cl. 514/12; 536/24.3

(57) ABSTRACT

The present invention relates to the identification of a specific inhibitor of the extrinsic clotting pathway from triatomine bugs, which we have designated TEPI (triatomine extrinsic pathway inhibitor). In one embodiment, the polypeptide inhibitor or fragment has a N-terminal sequence:

A-Met-Val-Thr-Asn-X-Asn-Met-Pro-Asn-Pro-Met-Thr-
Gly-Phe-Glu-Lys-Ser-X-Phe-Phe-Thr-X-Met-Trp-Tyr-
Val-

wherein X indicates any amino acid sequence residue or pharmaceutically acceptable salt thereof and A represents Ser, Asp or Glu, and preferably Ser.

### FIELD OF THE INVENTION

The present invention relates to anticoagulant proteins, and in particular to novel anticoagulants derived from triatomine bugs and their uses.

### BACKGROUND TO THE INVENTION

Blood coagulation involves the interplay of numerous plasma derived enzymes and cofactors. It can be initiated by two separate mechanism, either through the release or exposure of tissue factor into the blood, commonly termed the “extrinsic pathway”, or through the activation of the contact factors of plasma, commonly termed the “intrinsic pathway”. Both initiation pathways converge to a common pathway at a point where the prothrombinase complex catalyses the conversion of prothrombin to thrombin. Once formed thrombin cleaves soluble fibrinogen to insoluble fibrin which then crosslinks to form a blood clot.

The primary mechanism of blood coagulation in vivo is thought to involve the extrinsic pathway (Camerer, E. et al., Thromb. Res. 81: 1-41 (1996)). Here exposed membrane bound tissue factor (TF) acts as the primary trigger binding plasma Factor VIIa (VIIa) to form a TF-VIIa complex which then activates Factor 1× and X to Factor IXa and Xa respectively (FIG. 1). Factor Xa can then produce small amounts of thrombin via the prothrombinase complex, commonly termed the “initiation phase” of thrombin generation. Once formed, thrombin together with factor Xa then activates small quantities of plasma Factor VIII and V to Factor VIIIa and Va respectively, which in turn form two most efficient catalyst complexes. These are the tenase complex (IXa-VIIIa-Ca²⁺-phospholipids), which converts factor X to Xa, and the prothrombinase complex (Xa-Va-Ca²⁺-phospholipids), which converts the majority of prothrombin to thrombin, commonly termed the “propagation phase” of thrombin generation.

A number of physiological inhibitors of the extrinsic pathway have been identified. The most significant of these is tissue factor pathway inhibitor (TFPI), also known as extrinsic pathway inhibitor (EPI) or lipoprotein associated coagulation inhibitor (LACI). TFPI first forms a complex with Factor Xa which then binds to TF-VIIa complex resulting in a quartenary complex to limit production of IXa and Xa, thereby quenching the initiation phase of thrombin generation. Alternatively, at high concentrations, TFPI may also inhibit TF-VIIa complex directly in the absence of Factor Xa. Once this happens, Factor Xa can only be produced by the intrinsic pathway, namely via the tenase complex.

TFPI has been isolated and cloned. The protein has molecular weight of 34 kDa and the N-terminal sequence Asp-Ser-Glu-Glu-Asp-Glu-Glu-His-Thr-Ile-Ile-Thr-Asp-Thr-Glu-Leu-Pro-Pro-Leu-Lys-Leu. Analysis of the secondary structure indicates that TFPI possesses three kunitz-type inhibitor domains. Whereas domain I (from amino acid 22 to 79) is known to bind TF-VIIa and domain II (from amino acid 93 to 150) is known to bind Factor Xa, the function of domain III (from amino acid 185 to 242) remains unclear.

Other molecules suggested to inhibit the extrinsic pathway include TFPI-2, antithrombin III, Annexin V and platelet factor 4 (PF4). TFPI-2 is a protein with a predicted molecular mass of 24.6 kDa, which has three Kunitz-type domains, and shares sequence homology with

TFPI, but has been reported not to cross-react with an antiserum raised against TFPI. It is capable of inhibiting the amidolytic activities of human trypsin and TF-VIIa, but does not inhibit thrombin (Sprecher C. A. et al., Proc Natl Acad Sci USA 91: 3353-3357 (1994)).

Annexin V, also known as placental anticoagulant protein, has a molecular weight of approximately 37 kDa, binds TF in a Ca²⁺ dependent manner, and inhibits activation of Factor IX and Factor X by TF-VIIa, but has no effect on the amidolytic activity of Factor Xa (Kondo S. et al. Thromb Res 48: 449-459 (1987)). Its activity was not affected by an antiserum directed against TFPI.

Antithrombin III is a serine protease inhibitor which is abundant in human plasma. It is capable of inhibiting a number of coagulatory factors, including thrombin, Factor IXa and Factor Xa, but has also been shown to inhibit TF-VIIa in the presence of heparin (Rao L. V. M. et al. Blood 81: 2600-2607 (1993)).

A number of anticoagulant factors have been isolated from haematophagous insects, which use such factors to prevent clot formation during feeding and subsequent digestion of the blood meal. Triatomine bugs are the largest of the blood sucking insects (Lent, H. and Wygodzinsky, P., Revision of the Triatominae (Hemiptera, Reduviidae) and their significance as vectors of Chaga's disease (1979)). They can ingest appreciable quantities of blood during feeding due to hinged dorsal and ventral abdominal connexival plates. Early instar nymphs gorge themselves and can ingest up to 12 times their unfed body weight in blood whilst adult bugs rarely take more than 3 times their unfed body weight (Buxton, P. A. Trans. R. Ent. Soc. London 78: 227-236 (1930)).

In the mid 1960's researchers observed that salivary gland extracts of the triatomine bug, Rhodnius polixus delayed coagulation of horse plasma by the intrinsic pathway and named the product prolixin-S (Hellmann, K. and Hawkins, R., Nature 207: 265-267 (1965); GB 1,092,421). Prolixin-S is a hemeprotein with molecular weight of approximately 20 kDa which has been variously claimed to inhibit Factor VIII (Ribiero, J. M. C. et al., Biochem. J. 308: 243-249 (1995); Champagne, D. E. et al., J. Biol. Chem. 270: 8691-8695 (1995)) and Factor IXa-catalysed activation of Factor X, i.e. to be an inhibitor of the tenase complex, (Sun, J. et al., Thromb. Haemostas. 75: 573-577 (1996); Sun, J. et al., Insect Biochem. Mol. Biol. 28: 191-200 (1998); Zhang, Y. et al., Biochemistry 37: 10681-10690 (1998); JP 9,067,396A; JP 10,265,497A). In addition, a number of thrombin inhibitors have been identified in triatomine bugs including rhodniin from Rhodnius prolixus (Friedrich, T. et al. J. Biol. Chem. 268: 16216-16222 (1993); U.S. Pat. No. 5,523,287), triabin from Triatoma pallidipennis (Noeske-Jungblut, B. et al., J. Biol. Chem. 270: 28629-28634 (1995); U.S. Pat. No. 5,876,971) and dipetalogastin from Dipetalogaster maximus (Mende, K. et al., Eur. J. Biochem. 266: 583-590 (1999); DE 195,04,776A1).

### SUMMARY OF THE INVENTION

Broadly, the present invention is based on the identification of a specific inhibitor of the extrinsic clotting pathway from triatomine bugs, which we have designated TEPI (triatomine extrinsic pathway inhibitor).

Accordingly, in a first aspect, the present invention provides a polypeptide inhibitor of the extrinsic clotting pathway, or a fragment thereof, as obtainable from triatomine bugs.

In one embodiment, the polypeptide inhibitor or fragment has a N-terminal sequence:

A-Met-Val-Thr-Asn-X-Asn-Met-Pro-Asn-Pro-Met-Thr-
Gly-Phe-Glu-Lys-Ser-X-Phe-Phe-Thr-X-Met-Trp-Tyr-
Val-

wherein X indicates any amino acid sequence residue or pharmaceutically acceptable salt thereof and A represents Ser, Asp or Glu, and preferably Ser.

Preferably, the polypeptide inhibitor has a molecular mass of about 20 kDa as determined by SDS-PAGE.

Exposure of TF to circulating blood triggers blood coagulation and subsequent thrombus formation which may lead to various thrombotic disorders, such as acute myocardial infarction (AMI), deep vein thrombosis (DVT), disseminated intravascular coagulation (DIC), pulmonary embolism (PE), etc. Currently, these diseases are normally treated with various thrombolytic agents and/or anticoagulants like unfractionated heparin, low molecular weight heparin, hirudin and hirudin analogues. These adjunct anticoagulants are either direct or indirect inhibitors of preformed thrombin. Thus, a specific inhibitor of the extrinsic pathway offers an alternative and potent anticoagulant as it can prevent the initiation of thrombin formation by inhibiting complex formation higher up the coagulation cascade. It could therefore have major beneficial implications in the treatment and management of thrombotic disorders and also in the prevention of undesirable clotting, such as rethrombosis after successful thrombolysis during AMI.

Thus, the present invention also relates to the use of these inhibitors for medical or therapeutic purposes, and in a further aspect provides the above inhibitors of the extrinsic clotting pathway as obtainable from triatomine bugs for use in a method of medical treatment.

In a further aspect, the present invention provides the use of an inhibitor of the extrinsic clotting pathway obtainable from triatomine bugs in the preparation of a medicament for the treatment of a disorder characterised by the abnormal or undesirable activation of the extrinsic clotting pathway.

The invention additionally relates to compositions comprising the above inhibitors. Such compositions may be pharmaceutical compositions comprising an inhibitor according to the present invention, optionally in combination with one or more pharmaceutically acceptable excipients or carriers.

In another aspect, the present invention describes an antibody capable of binding to an inhibitor of the extrinsic clotting pathway obtainable from triatomine bugs. Exemplary antibodies include polyclonal, monoclonal, humanized, bispecific and heteroconjugate antibodies, the production of which is well known in the art.

In a further aspect, the present invention provides a method for isolating an inhibitor of the extrinsic pathway obtainable from triatomine bugs.

The present invention also relates to a nucleic acid molecule encoding one of the above defined polypeptide inhibitors of the extrinsic clotting pathway obtainable from triatomine bugs. In one embodiment, the invention relates to a nucleic acid probe which comprises a sequence which encodes all or part of a polypeptide sequence as set out in:

A-Met-Val-Thr-Asn-X-Asn-Met-Pro-Asn-Pro-Met-Thr-
Gly-Phe-Glu-Lys-Ser-X-Phe-Phe-Thr-X-Met-Trp-Tyr-
Val-

wherein X indicates any amino acid sequence residue or pharmaceutically acceptable salt thereof and A represents Ser, Asp or Glu, and preferably Ser.

In a further aspect, the present invention details an expression vector comprising the nucleic acid encoding the above defined polypeptide inhibitors and host cells transformed with the expression vector. The present invention also includes a method of producing a polypeptide inhibitor, the method comprising culturing the above defined host cells and isolating the polypeptide produced. Having expressed the polypeptide, the method may comprise the additional step of formulating it in a composition, e.g. by admixing it with a suitable carrier.

Also described is a method for isolating a nucleic acid molecule encoding an inhibitor of the extrinsic clotting pathway from triatomine bugs. In one embodiment, this method may comprise the step of probing a suitable library with a probe as described above. In a yet further aspect, the invention provides nucleic acid molecules encoding inhibitors of the extrinsic clotting pathway obtainable from triatomine bugs.

Embodiments of the present invention will now be described by way of example and not limitation with reference to the accompanying drawings.

### BRIEF DESCRIPTION OF THE DRAWINGS

FIG. 1 shows a schematic diagram of the blood coagulation mechanism. The initiation phase provides small quantities of thrombin via the prothrombinase complex thereby enabling feedback activation of the tenase and prothrombinase complex and subsequent rapid conversion of the majority of prothrombin to thrombin.

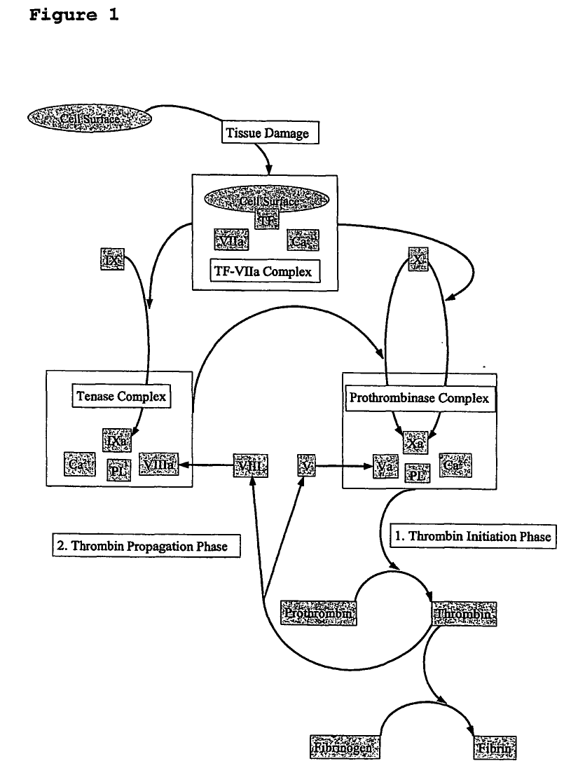

FIG. 2 shows the concentration dependent effect of crude salivary gland extract of Dipetalogaster maximus on the prothrombin time (PT), activated partial thromboplastin time (APTT) and thrombin clotting time (TCT).

FIG. 3 shows the effect of salivary gland extract separated by ultrafiltration into varying molecular weight fractions on plasma clotting times. TEPI appears in all filtrates except the 10 kDa filtrate indicating a molecular weight of between 10-30 kDa.

FIG. 4 shows the purification of TEPI by reverse phase HPLC. The bar indicates the elution of TEPI.

FIG. 5 shows SDS-PAGE and Western blot of purified TEPI. The band on the SDS-PAGE indicates TEPI has a molecular weight of approximately 20 kDa under reduced conditions. The Western blot data indicates that a polyclonal antibody raised against native human full length TFPI recognizes TEPI.

FIG. 6 shows mass spectrometry data of purified TEPI.

FIG. 7 shows the results of an affinity binding experiment and shows that TEPI binds to Factor Xa but not to thrombin.

### DETAILED DESCRIPTION

The term “inhibitor of the extrinsic clotting pathway”, is herein defined as a factor capable of increasing the prothrombin time (PT), but having little or no effect on the activated partial thromboplastin time (APTT), as determined below (Examples). By this is meant that a blood plasma concentration of inhibitor which provides a 100% increase in prothrombin time (PT) provides less than 15% change (either increase or decrease) in activated partial thromboplastin time (APTT), and preferably less than 10% change (either increase or decrease) in activated partial thromboplastin time (APTT). Such factors are hereinafter referred to as “inhibitors” and include the polypeptide inhibitors identified herein from triatomines and functional fragments thereof.

The prothrombin time (PT) test and the activated partial thromboplastin time (APTT) test are standard assays, well known to those skilled in the art, which enable the effects of factors on the extrinsic and intrinsic clotting pathways to be differentiated. In the prothrombin time (PT) test tissue factor is added to plasma so that activation proceeds through the extrinsic pathway. In the activated partial thromboplastin time (APTT) test blood plasma is activated by contact factors, such as kaolin or glass. Where products affect coagulation after convergence of the two pathways the activated partial thromboplastin time (APTT) test is favoured since it is more sensitive than the prothrombin time (PT) test to inhibitors of the common pathway. Thus, prolongation of the prothrombin time (PT) but not the activated partial thromboplastin time (APTT) shows that a factor acts to inhibit the extrinsic pathway upstream of the convergence point of the intrinsic and extrinsic pathways.

Typically, the inhibitor of the present invention also has little or no effect on the thrombin clotting time (TCT), in which thrombin is added to blood plasma.

By “triatomine” is meant any protostomia that is an obligate vertebrate bloodsucker, for example those insects within the families Hemiptera and Reduviidae. Suitable genera of insects may include Triatoma, Rhodnius, Dipetalogaster, Panstrongylus, Eratyrus, Alberprosenia, Belminus, Microtriatoma, Parabelminus, Cavernicola, Psammolestes, Linshcoteus and Paratriatoma. In specific embodiments, suitable sources of inhibitors according to the present invention may include triatomines of epidemiological significance such as Triatoma infestans, T. dimidiata, T. phyllosoma, T. pallidipennis, T. sordida, T. brasiliensis, T. guasayana, T. patagonica, T. maculata, T. carrioni, Rhodnius prolixus, R. pallescens, R. ecuadoriensis, Panstrongylus megistus, P. rufotuberculatus, P. chinai, or P. herreri and less commonly Triatoma barberi, T. rubida, T. sanguisuga, T. lecticularia, T. protracta, Eratyrus mucronatus and Panstrongylus geniculatus as well as other non domesticated triatomines including Alberprosenia goyovargasi, Belminus costaricensis, Microtriatoma trinidadensis, Parabelminus yurupucu, Cavernicola pilosa, Psammolestes arthuri, Rhodnius brethesi, Dipetalogaster maximus, Linshcoteus confumus and Paratriatoma hirsuta.

Thus, the present invention provides an inhibitor of the extrinsic clotting pathway as obtainable from triatomine bugs. In one embodiment, the inhibitor is as obtainable from a triatomine of the genus Dipetalogaster. In a further embodiment, the inhibitor is as obtainable from Dipetalogaster maximus.

The protein designated TEPI was isolated from the salivary glands of Dipetalogaster maximus by reverse phase HPLC. The isolated protein was shown by SDS-PAGE to have a molecular weight of approximately 20 kDa. This was confirmed to be 19.893 kDa by mass spectroscopy. Automated sequence analysis of the protein provided the N-terminal sequence

A-Met-Val-Thr-Asn-X-Asn-Met-Pro-Asn-Pro-Met-Thr-
Gly-Phe-Glu-Lys-Ser-X-Phe-Phe-Thr-X-Met-Trp-Tyr-
Val-

Wherein X indicates any amino acid sequence residue or pharmaceutically acceptable salt thereof and A represents Ser, Asp or Glu, and preferably Ser. Although this sequence has no homology to human TFPI, immunoblotting showed that TEPI cross reacts with a rabbit antiserum raised against human TFPI.

Pharmaceutical Compositions

In one aspect of the present invention, TEPI polypeptides can be used for the treatment of disorders characterised by the abnormal or undesirable activation of the extrinsic clotting pathway. Such disorders include acute myocardial infarction (AMI), deep vein thrombosis (DVT), disseminated intravascular coagulation (DIC), and pulmonary embolism (PE), as well as rethrombosis after successful thrombolysis during AMI.

The polypeptides of the invention can be formulated in pharmaceutical compositions. These compositions may comprise, in addition to one of the above substances, a pharmaceutically acceptable excipient, carrier, buffer, stabiliser or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil; Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

Compositions comprising TEPI according to the present invention may be administered alone or in combination with other anticoagulant or thrombolytic treatments, either simultaneously or sequentially dependent upon the condition to be treated.

Antibodies

The invention further describes antibodies capable of binding to inhibitors according to the present invention. The anti-inhibitor antibodies may comprise polyclonal antibodies. Methods of preparing polyclonal antibodies are known to the skilled artisan. Polyclonal antibodies can be raised in a mammal for example, by one or more injections of an immunizing agent and, if desired, an adjuvant. Typically, the immunizing agent and/or adjuvant will be injected in the mammal by multiple subcutaneoues or intraperitoneal injections. The immunizing agent may include an inhibitor polypeptide or a fusion protein thereof. It may be useful to conjugate the immunizing agent to a protein known to be immunogenic in the mammal being immunized. Examples of such immunogenic proteins may include but are not limited to keyhole limpet hemocaynin, serum albumin, bovine thyroglobulin, and soybean trypsin inhibitor. Examples of adjuvants which may be employed include Freund's complete adjuvant and MPL TDM adjuvant. The immunization protocol may be selected by one skilled in the art without undue experimentation.

The anti-TEPI antibodies may, alternatively, be monoclonal antibodies. Monoclonal antibodies may be prepared using hybridoma methods, such as those described by Kohler and Milstein (Nature 256: 495 (1975)). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include the inhibitor polypeptide or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylenen glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phophoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Rockville, Md. Human myeloma and mouse-human heteromyeloma cell lines also have been described for the production of human monoclonal antibodies (Kozbor, J Immunol, 133: 3001 (1984); Brodeur et al., Monoclonal Antibody Production Techniques and Applications Marcel Dekker, Inc., New York, (1987) pp. 51-63).

The culture medium in which the hybridoma cells are cultured can then be assayed for the presence of monoclonal antibodies directed against inhibitor polypeptides. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, (Anal Biochem 107:220 (1980)).

After the desired hybridoma cells are identified, the clones may be subcloned by limiting dilution procedures and grown by standard methods (Goding, supra). Suitable culture media for this purpose include, for example, Dulbecco's Modified Eagle's Medium and RPMI-1640 medium. Alternatively the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain cross-linking. Alternatively, the relevant cysteine residues are substituted with another amino acid residue or are deleted so as to prevent cross-linking.

In vitro methods are also suitable for preparing monovalent antibodies. Digestion of antibodies to produce fragments thereof, particularly, Fab fragments, can be accomplished using routine techniques known in the art.

Methods of Isolating Inhibitor Polypeptides

Inhibitors according to the present invention may be isolated from tissues of suitable insect species. Particularly suitable tissues are salivary glands, and gut. Suitable techniques for protein isolation and purification are well known to those skilled in the art. By way of example, target proteins may be prepared from tissue homogenates prepared by standard methods, or as herein described (see the Examples). Homogenates may be subjected to an initial fractionation, for example on the basis of molecular weight (e.g. by ultrafiltration) or salt solubility (e.g. buy ammonium sulphate precipitation), and individual fractions tested for extrinsic pathway inhibitor activity. Inhibitor polypeptides may then be purified from positive fractions on the basis of size, charge, or solubility by known separation techniques such as gel filtration, ion exchange chromatography, hydrophobic interaction chromatography, HPLC, or reveresed phase HPLC.

Additionally or alternatively, the purification process may involve affinity chromatography methods. Such purification methods typically employ binding agents having binding sites capable of specifically binding to the desired polypeptides, or fragments thereof in preference to other molecules. Examples of binding agents include antibodies, receptors and other molecules capable of specifically binding the analyte of interest. Suitable binding agents are antibodies raised against, or capable of binding to, inhibitors according to the present invention, such as antibodies against human TFPI. Alternative binding agents are physiological binding partners of inhibitors according to the present invention, such as components of the clotting cascade.

The sample is generally contacted with the binding agent(s) under appropriate conditions which allow the analyte in the sample to bind to the binding agent(s). Conveniently, the binding agents are immobilised on solid supports to facilitate purification. Alternatively, the sample may subsequently be contacted with an immobilised second binding agent capable of binding the first binding agent or a label or tag attached thereto, such as biotin. Examples of suitable second binding agents are agents capable of binding immunoglobulins, such as anti-Ig antibodies, Protein A or Protein G, antibodies directed against the first binding agent, and agents with affinity for labelling molecules present on the first binding agent, such as streptavidin, which forms a high-affinity complex with biotin. Contaminating components can then be washed away from the immobilised target polypeptides, and the target polypeptides eluted as appropriate.

Nucleic Acid Encoding Extrinsic Pathway Inhibitors

On the basis of the sequence information provided herein, the skilled person is able to isolate nucleic acids coding for inhibitors according to the present invention. Amino acid sequence information may be used to design oligonucleotide probes or primers, taking into account the degeneracy of the genetic code, and where appropriate, codon usage of the organism from the candidate nucleic acid is derived. Expression of the polypeptide inhibitor nucleic acid provides a convenient way of producing large amounts of the polypeptide.

Such oligonucleotide probes may be used to probe suitable libraries, such as poly(A)-selected mRNA, cDNA or genomic DNA libraries from suitable oraganisms/tissues, in order to isolate nucleic acid molecules encompassing full-length coding sequences for inhibitors according to the present invention. Nucleic acid isolated and/or purified from one or more cells or a nucleic acid library derived from nucleic acid isolated and/or purified from cells (e.g. a cDNA library derived from mRNA isolated from the cells), may be probed under conditions for selective hybridization and/or subjected to a specific nucleic acid amplification reaction such as the polymerase chain reaction (PCR).

The conditions of a hybridization reaction can be controlled to minimise non-specific binding, and preferably stringent to moderately stringent hybridization conditions are preferred. The skilled person is readily able to design such probes, label them and devise suitable conditions for the hybridization reactions, assisted by textbooks such as Sambrook, Maniatis and Fritsch, Molecular Cloning, A Laboratory Manual, Cold Spring Harbor Laboratory Press (1989), and Ausubel et al, Short Protocols in Molecular Biology, John Wiley and Sons (1992). Binding of a probe to target nucleic acid may be measured using any of a variety of techniques at the disposal of those skilled in the art. For instance, probes may be radioactively, fluorescently or enzymatically labelled. Other methods not employing labelling of the probe include examination of restriction fragment length polymorphism, amplification using PCR, RNAse cleavage and allele specific oligonucleotide probing. Hybridization is generally followed by identification of successful hybridization and isolation of nucleic acid which has hybridized to the probe, which may involve one or more steps of PCR.

Nucleic acid amplification techniques may also be used to obtain nucleic acids according to the present invention. One or more oligonucleotide probes or primers may be designed to hybridize with nucleic acids encoding all or part of the amino acid sequence shown herein, as set out above, particularly with fragments of relatively rare sequence, based on codon usage or statistical analysis. A primer designed to hybridize with a fragment of the target nucleic acid sequence may be used in conjunction with one or more oligonucleotides designed to hybridize to a sequence in a cloning vector within which target nucleic acid has been cloned, or in so-called “RACE” (rapid amplification of cDNA ends) in which cDNA's in a library are ligated to an oligonucleotide linker and PCR is performed using a primer which hybridizes with the known target sequence and a primer which hybridizes to the oligonucleotide linker. An oligonucleotide for use in nucleic acid amplification may have about 10 or fewer codons (e.g. 6, 7, or 8), i.e. be about 30 or fewer nucleotides in length (e.g. 18, 21 or 24). Generally specific primers are upwards of 14 nucleotides in length, but not more than 18-20. Those skilled in the art are well versed in the design of primers for use in processes such as PCR. (For appropriate methods and principles, see “PCR protocols; A guide to Methods and Applications”, Eds. Innis et al, Academic Press, New York, (1990)).

In order to generate a full-length coding sequence, it may be necessary for one or more gene fragments to be ligated together. Where a full-length coding sequence has not been obtained in a single molecule, a smaller molecule representing part of the full molecule may be used to obtain full length clones. The full-length clones isolated may be sequenced, or be subcloned into expression vectors and activity assayed by transfection into suitable host cells. The presence of a TEPI or related peptide may be detected, for example, by appropriate activity assays or use of suitable antibodies, such as a polyclonal antiserum directed against human TFPI, or a specific antibody raised against TEPI.

Probes derived from nucleic acids encoding inhibitors according to the present invention may be used to screen libraries derived from other species, in order to identify other related inhibitors. Such probes will typically correspond to highly conserved regions of the protein, such as those necessary for binding and activity.

### EXAMPLE 1

### Rearing of Triatomines

Triatomine bugs of the genus Dipetalogaster were reared in the laboratory. Colonies were maintained at 28° C., 60-70% relative humidity on a 14 hour/10 hour day/night cycle. At one weekly intervals insects were allowed to feed on the breast of live chickens until satiated. The time taken for eggs to develop, via the 1st, 2nd, 3rd, 4th and 5th instar, to adulthood took 6 to 8 months. Adults males measuring between 34-40 mm in length were used in the preparation of TEPI.

### EXAMPLE 2

### Isolation of Triatomine Salivary Glands and Extraction of Anticoagulant

The salivary glands (D1 glands according to Barth, R. Mem. Inst. Oswaldo Cruz, 52: 517-587 (1954)) of Dipetalogaster maximus, located in the anterior portion of the thorax, were dissected under a stereomicroscope by pinning the bug in a dissecting dish lined with wax and covered with 10 mM Tris-HCl, 0.15 M NaCl, pH 7.4. The wings and pronotum were removed together with the musculature of the wings to reveal the paired salivary glands. Each salivary gland had an approximate volume of 1.8 ul.

In an initial experiment (A) the paired salivary glands were removed and homogenised in 100 ul 20 mM Tris-HCl, 0.15 M NaCl, pH 7.4 and frozen at −70° C. In a second experiment (B) the salivary glands were removed and homogenised in 100 ul buffer as above and the homogenate centrifuged at 30,000 rpm for 5 minutes. The supernatant was removed and frozen at −70° C. and the pellet re-suspended in 100 ul 20 mM Tris-HCl, 0.15 M NaCl, pH 7.4 buffer and frozen at −70° C.

In a third experiment (C) the paired salivary glands we-re removed and homogenised in 100 ul 20 mM Tris-HCl, 0.15 M NaCl, pH 7.4 containing 2 mM CHAPS (3-[(3-cholamidopropyl)-dimethylammonio]-1-propanesulphonate) and the homogenate centrifuged at 30,000 rpm for 5 minutes. The supernatant was removed and frozen at −70° C. and the pellet re-suspended in 100 ul 20 mM Tris-HCl, 0.15 M NaCl, pH 7.4 containing 2 mM CHAPS buffer and frozen at −70° C. The protein content of the supernatant was determined to be approximately 190 ug indicating that each salivary gland contained approximately 95 ug of protein. The protein concentration in the salivary gland was calculated to be 50 mg/ml.

### EXAMPLE 3

### Effect of Salivary Gland Extract on Plasma Clotting Times

The thrombin clotting time (TCT), prothrombin time (PT) and activated partial thromboplastin time (APTT) were determined by adding 100 ul salivary gland extract harvested according to experiment (A), (B) or (C) from Example 2 to 400 ul normal control plasma (0.4% v/v salivary gland concentration) and incubating for 5 minutes at 37° C. In order to provide qualitative data the clotting tests were determined manually in 10×75 mm borosilicate glass tubes.

For TCT determination 100 ul plasma/extract was added to 100 ul 50 mM imidizole buffer pH 7.4 for 30 seconds at 37° C. Then 50 ul pre-warmed human thrombin (5 NIH units/ml initial concentration diluted to obtain a precise control clotting time of 15 seconds) was added and the time taken for clot formation to occur was recorded using a stopwatch.

For PT determination 50 ul pre-warmed rabbit brain thromboplastin was added to 50 ul plasma/extract and incubated for 1 minute at 37° C. Then 50 ul pre-warmed 25 mM CaCl₂ was added to the mixture and the time taken for clot formation to occur was recorded using a stopwatch.

For APTT determination 200 ul kaolin/platelet substitute was added to 100 ul plasma/extract and incubated for 2 minutes at 37° C. Then 100 ul pre-warmed 25 mM CaCl₂ was added to the mixture and the time taken for clot formation to occur was recorded using a stopwatch.

Results were expressed as ratios of the test clotting time versus the control clotting time in which 100 ul 10 mM Tris-HCl, 0.15 M NaCl, pH 7.4 buffer (experiment A and B) or 100 ul 20 mM Tris-HCl, 0.15 M NaCl, pH 7.4 containing 2 mM CHAPS buffer was substituted for salivary gland extract (experiment C).

Table 1 shows the effect of the salivary gland extracts from experiments (A), (B) and (C) from Example 2 on the prothrombin time, activated partial thromboplastin time and thrombin clotting time. The results indicate that the addition of 2 mM CHAPS to the buffer improves the extraction of TEPI into the supernatant.

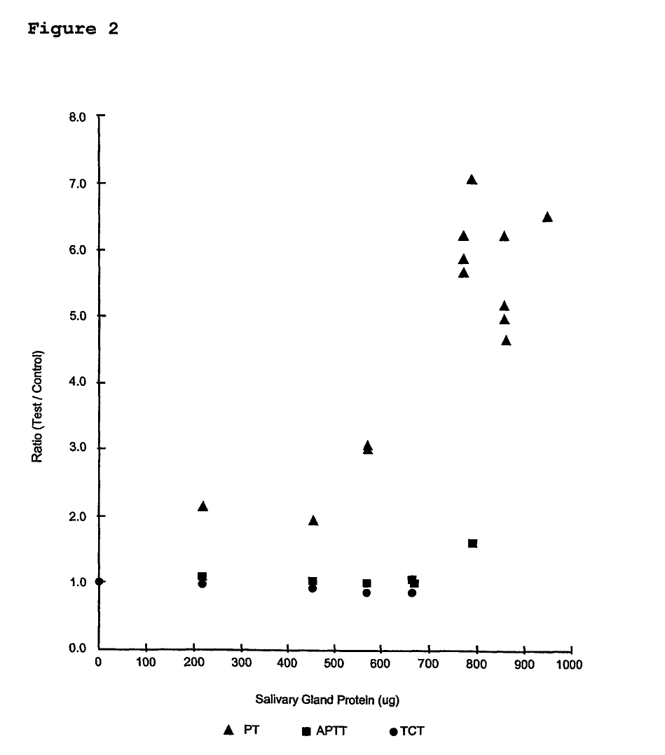

FIG. 2 shows the effect of varying concentrations of salivary gland material extracted in the same way as described in the preparation of the supernatant (Example 2, experiment (C)) on plasma clotting times.

TABLE 1

| | PTT | | APTT | | TCT | |
|---|---|---|---|---|---|---|
| Sample | Time (s) | Ratio | Time (s) | Ratio | Time (s) | Ratio |
| Tris buffer | 16 | 1.00 | 46 | 1.00 | 15 | 1.00 |
| control | | | | | | |
| (A) Whole | 34 | 2.13 | 49 | 1.07 | 15 | 1.00 |
| extract | | | | | | |
| (B) Supernatant | 25 | 1.56 | 47 | 1.02 | 16 | 1.07 |
| (B) Pellet | 29 | 1.81 | 46 | 1.00 | 15 | 1.00 |
| Tris CHAPS | 15 | 1.00 | 47 | 1.00 | 16 | 1.00 |
| buffer control | | | | | | |
| (C) Supernatant | 35 | 2.33 | 48 | 1.02 | 15 | 0.93 |
| (C) Pellet | 19 | 1.27 | 45 | 0.96 | 14 | 0.88 |

### EXAMPLE 4

### Separation of Salivary Gland Extract into Molecular Weight Fractions by Ultrafiltration

A homogenate of 10 salivary glands (D1) was prepared in 20 mM Tris-HCl, 0.15 M NaCl, pH 7.4 containing 2 mM CHAPS buffer in the same way as described in the preparation of the supernatant in Example 2 experiment (C). The pellet was extracted three times and the supernatants combined. The supernatant was separated into five equal portions and ultrafiltered through a 0.22 um or four varying molecular weight cut-off filters (Microcon™, Millipore). The filtrate was tested for anticoagulant activity as described in Example 3.

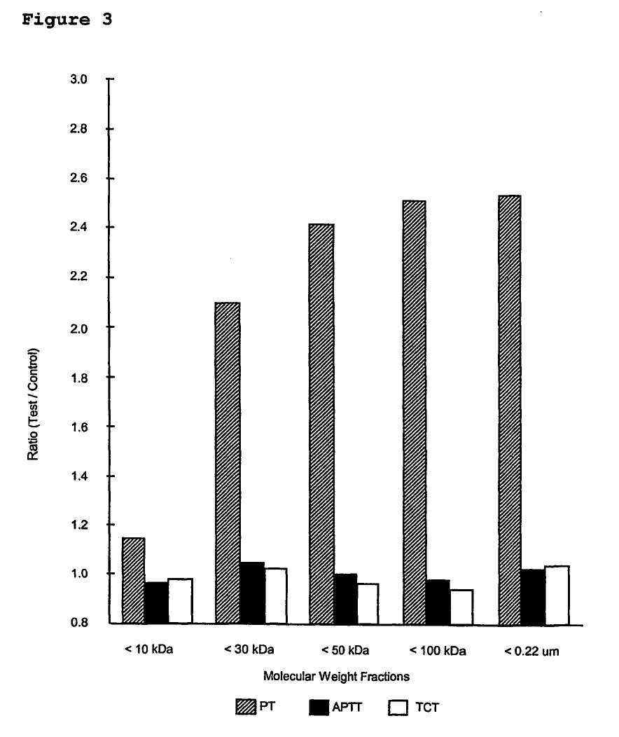

FIG. 3 shows the results from the ultrafiltration experiment. TEPI is able to pass through a 0.22 um filter as well as a 100, 50 and 30 membrane but not through a 10 kDa membrane suggesting a molecular weight of between 10 and 30 kDa.

### EXAMPLE 5

### Affinity Binding Experiments of TEPI to the Pivitol Coagulation Factors

Affigel-Xa (Biorad) was prepared according to the manufacturers recommendations using Affigel 15 and purified Factor Xa. Briefly, 1 ml Factor Xa (3 mg/ml in 20 mM Tris-HCl, 0.7M NaCl, pH 7.4) was dialysed overnight at 4° C. against 100 mM HEPES, pH 7.4 before adding to 0.75 ml of Affigel 15 prewashed in 10 mM sodium acetate, pH 4.5. The Factor Xa ligand was left to couple for 4 hours at 4° C. with regular agitation. The slurry was centrifuged and the supernatant assayed for residual Factor Xa. Results indicated that between 80-90% coupling was achieved. Binding of Factor Xa to affigel 15 was confirmed by chromogenic assay (S-2288). Affigel-Thrombin (Biorad) was prepared in the same way using Affigel 10 and purified Thrombin.

A concentrated homogenate of 20 salivary glands (D1) was prepared in 200 ul 20 mM Tris-HCl, 0.15 M NaCl, pH 7.4 containing 2 mM CHAPS buffer in the same way as described in the preparation of the supernatant in Example 2 experiment (C). The supernatant was filtered through a 0.22 um ultrafilter and then 100 ul added to either 400 ul Affigel-Xa or 400 ul Affigel-Thrombin, previously washed in 20 mM Tris-HCl, 0.15 M NaCl, pH 7.4 containing 2 mM CHAPS. The gel was mixed every 5 minutes for 30 minutes at room temperature prior to centrifugation. 100 ul of the supernatant was removed and added to 400 ul normal control plasma for assay of TCT, PT and APTT as described in Example 3.

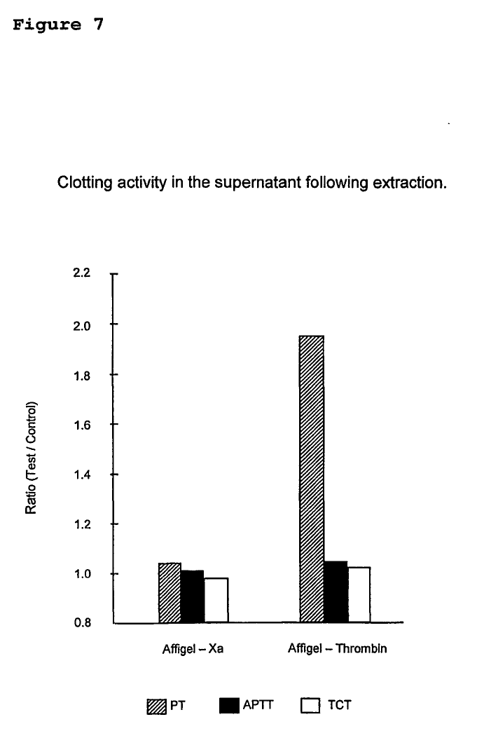

FIG. 7 shows the results from the affinity binding experiments. TEPI clearly binds to Factor Xa but not to Thrombin.

### EXAMPLE 6

### Purification of TEPI by Reverse Phase HPLC

A homogenate of 10 salivary glands (D1) was prepared in 0.5 ml 20 mM Tris-HCl, 0.15 M NaCl, pH 7.4 containing 2 mM CHAPS buffer in the same way as described in the preparation of the supernatant in Example 2 experiment (C). The supernatant was filtered through a 0.22 um ultrafilter before being applied to a 4.6×220 mm reverse phase column of Aquapore OD-300 equilibrated in 0.1% (v/v) trifluoroacetic acid, 30% (v/v) acetonitrile (Solvent A). Bound material was eluted from the column using a 0 to 100% linear gradient containing 0.085% (v/v) trifluoroacetic acid, 45% (v/v) acetonitrile (Solvent B) over 30 minutes. The eluent was monitored at 215 nm and fractions collected were dried by centrifugal evaporation and resuspended in 20 mM Tris-HCl, 0.15 M NaCl, pH 7.4 containing 2 mM CHAPS buffer. Fractions were tested for anticoagulant activity as described in Example 3. The retention time for TEPI under these conditions was 16 minutes. C₁₈ reverse phase HPLC allowed a 20 fold purification of TEPI with 38 ug of purified TEPI to be recovered from the 950 ug of salivary gland material applied to the column. Calculations indicate that TEPI represented approximately 4% of the total salivary gland protein.

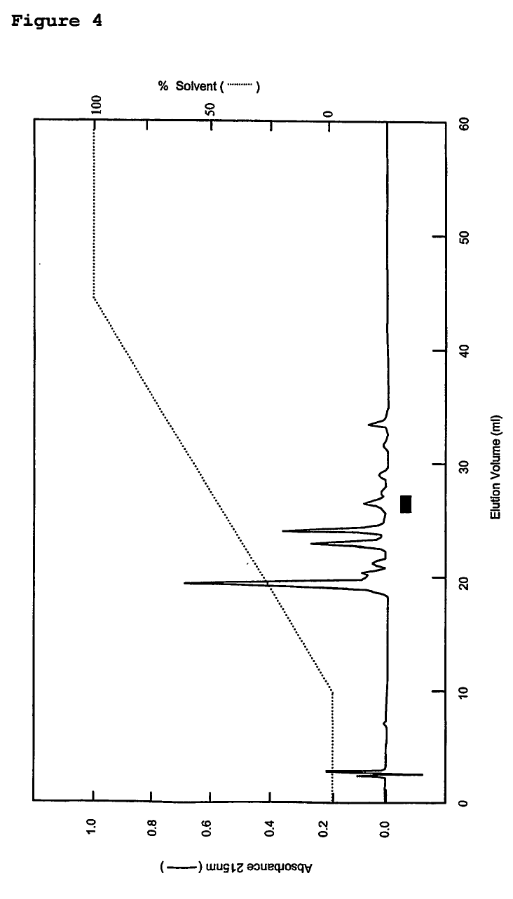

FIG. 4 shows the elution profile of the supernatant. The bar indicates the fractions which contain TEPI activity.

### EXAMPLE 7

### Molecular Weight Determination of TEPI

Fractions which contained TEPI activity were analysed by polyacrylamide gel electrophoresis (PAGE) in the presence of sodium dodecyl sulphate (SDS) to determine molecular weight. Vertical resolving gels of 1.5 mm thickness containing 12% (w/v) polyacrylamide were cast using the Laemmli buffer system (Laemmli, Nature 227: 680-685 1970). TEPI was loaded onto a 3% (w/v) polyacrylamide stacking gel. The gel was resolved under reducing conditions at constant current of 35 mA for about 70 minutes, stained with Coomassie blue and the apparent molecular weight of TEPI was determined by its relative mobility against standard proteins of known molecular weight.

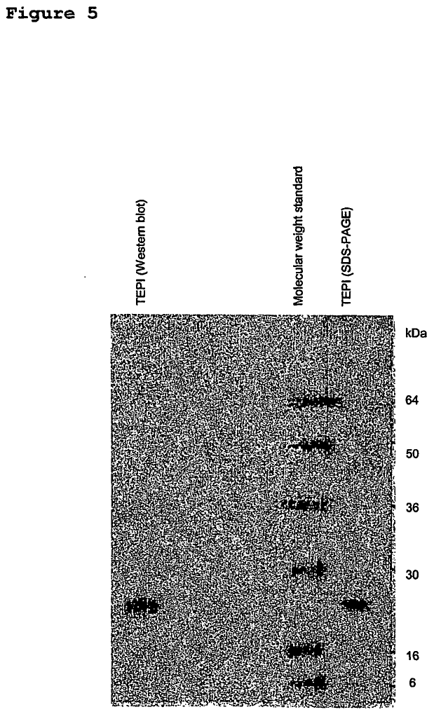

Under these conditions TEPI migrates on SDS-PAGE as a single band of approximately 20 kDa (FIG. 5).

### EXAMPLE 8

### Immunological Cross Reactivity of TEPI

Western blot analysis was performed to assess the immunological cross reactivity of TEPI with a polyclonal antibody against native human TFPI. SDS-PAGE of TEPI was carried out as described in Example 6. The gel was removed from its cassette and the proteins transferred onto a PVDF membrane by electroblotting. Briefly, the gel was sandwiched between two pieces of filter paper and the PVDF membrane on one side and two pieces of filter paper on the other side, all of them pre-soaked in electrotransfer buffer. The top and bottom electrodes were positioned in the blotting apparatus and electroblotted at a constant current of 80 mA for about 45 minutes. After blotting the membrane was removed and immersed in blocking buffer containing 1.0% (w/v) fish skin gelatin in phosphate buffered saline containing 0.1% Tween 20 (PBS-Tween) for 60 minutes. The membrane was removed, washed three times in PBS-Tween and soaked in the primary rabbit anti-human TFPI antibody (American Diagnostica Incorporated, Cat.# 4901) solution in gelatin-PBS-Tween with constant agitation for 60 minutes. The membrane was removed washed three times in PBS-Tween and then incubated with constant agitation in the secondary goat anti-rabbit antibody conjugated to horseradish peroxidase. After 60 minutes the membrane was washed with PBS and the band(s) visualised by soaking the membrane in diaminobenzidine solution containing H₂O₂ until the colour developed. The reaction was stopped by washing in water and the membrane allowed to dry in air.

The Western blotting results are shown in FIG. 5. The data indicate that TEPI cross reacts with anti-human TFPI antibody suggesting that the antibody recognizes an epitope(s) in TEPI which is also present in TFPI.

### EXAMPLE 9

### N-Terminal Protein Sequence of TEPI

N-terminal amino acid sequence analysis of TEPI was obtained by automated Edman degradation on an automated liquid pulse amino acid sequencer linked to an on-line analyser for identification of PTH amino acids.

Purified TEPI prepared by C₁₈ reverse phase HPLC as in Example 5 was adsorbed onto Prosorb membrane, washed, dried and then loaded onto the sequencer. Alternatively the appropriate band from the PDVF membrane as described in Example 6 was cut and analysed. Up to 27 amino acid residues from the N-terminus of TEPI were obtained as detailed.

A-Met-Val-Thr-Asn-X-Asn-Met-Pro-Asn-Pro-Met-Thr-
Gly-Phe-Glu-Lys-Ser-X-Phe-Phe-Thr-X-Met-Trp-Tyr-
Val-

wherein X indicates any amino acid residue and A represents Ser, Asp or Glu, but preferably Ser.

The above sequence has no sequence homology with human TFPI or other proteins when searched against SWISSPROT and PIR databases.

### EXAMPLE 10

### Analysis of TEPI by Mass Spectrometry

A Micromass TofSpec 2E mass spectrometer was used in positive ion mode to acquire data by Matrix Assisted Laser Desorbtion Ionization-Time of Flight (MALDI-TOF). Purified TEPI prepared by C₁₈ reverse phase HPLC as in Example 6 was reconstituted in 2 ul formic acid (80% v/v) and then a minute later was diluted with 2 ul water. This solution was mixed with sinapinic acid in a 1:1 ratio. An aliquot of 1 ul of this mixture was spotted onto a thin film of sinapinic acid which had previously been deposited onto the sample holder. The sample was analysed in a linear mode. External calibration was performed using horse heart myoglobin and bovine trypsinogen. A Database search was performed using the Protein Probe search engine.

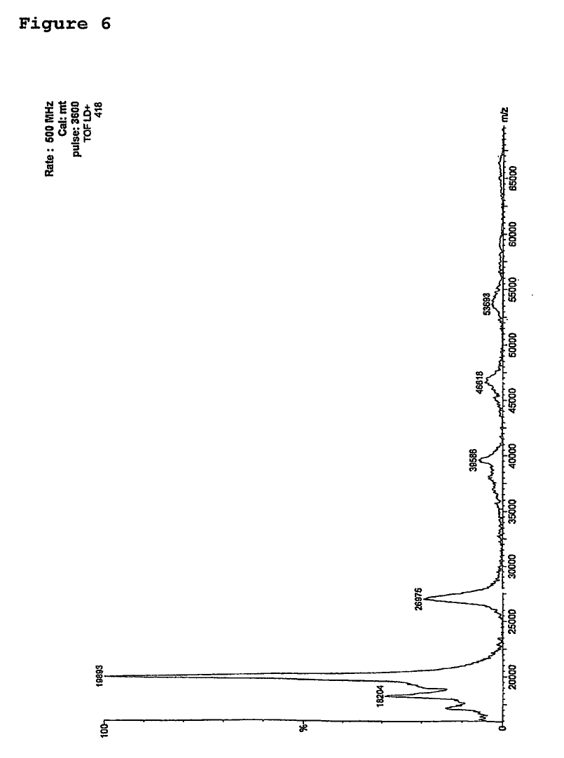

The mass spectrum (FIG. 6) showed one major peak at 19.893 kDa and two minor peaks at 18.204 kDa and 26.975 kDa with peak heights in the ratio of approximately 5:1:1 respectively. Several much smaller peaks at higher molecular mass were also observed. The database search of molecular mass gave no conclusive results.

The references mentioned herein are all expressly incorporated by reference.

1. A polypeptide inhibitor of the extrinsic clotting pathway, as obtainable from triatomine bugs, having N-terminal sequence:
A-Met-Val-Thr-Asn-X-Asn-Met-Pro-Asn-Pro-Met-Thr-Gly-Phe-Glu-Lys-Ser-X-Phe-Phe-Thr-X-Met-Trp-Tyr-Val-
wherein X indicates any amino acid sequence residue or pharmaceutically acceptable salt thereof and A represents Ser, Asp or Glu, and preferably Ser.

2. The polypeptide inhibitor of claim 1, wherein the polypeptide inhibitor is as obtainable from a triatomine bug of the genus Dipetalogaster.

3. The polypeptide inhibitor of claim 1 or claim 2, wherein the polypeptide inhibitor is as obtainable from Dipetalogaster maximus.

4. The polypeptide inhibitor of any one of the preceding claims, wherein the polypeptide inhibitor has a molecular mass of about 20 kDa as determined by SDS-PAGE.

5. The polypeptide inhibitor of any one of the preceding claims, wherein the inhibitor is capable of increasing the prothrombin time (PT) and has substantially no effect on the activated partial thromboplastin time (APTT).

6. The polypeptide inhibitor of any one of the preceding claims, wherein the inhibitor has substantially no effect on the thrombin clotting time (TCT).

7. A polypeptide inhibitor of any one of claims 1 to 6 for use in a method of medical treatment.

8. Use of a polypeptide inhibitor of any one of claims 1 to 6 for the preparation of a medicament for the treatment of a thrombotic disorder.

9. The use of claim 8, wherein the thrombotic disorder is acute myocardial infarction (AMI), deep vein thrombosis (DVT), disseminated intravascular coagulation (DIC), pulmonary embolism (PE), or rethrombosis after successful thrombolysis during AMI.

10. A composition comprising a polypeptide inhibitor of any one of claims 1 to 6.

11. An isolated nucleic acid molecule encoding a polypeptide inhibitor of any one of claims 1 to 6.

12. An expression vector comprising a nucleic acid molecule of claim 11, operably linked to sequences to control its expression.

13. A host cell transformed with the expression vector of claim 12.

14. A method of producing a polypeptide inhibitor of any one of claims 1 to 6, the method comprising culturing the host cells of claim 13 and isolating the polypeptide thus produced.

15. The method of claim 14, further comprising formulating the polypeptide in a composition by admixing it with a carrier.

16. A nucleic acid probe which comprises a sequence which encodes a polypeptide sequence as represented by:
A-Met-Val-Thr-Asn-X-Asn-Met-Pro-Asn-Pro-Met-Thr-Gly-Phe-Glu-Lys-Ser-X-Phe-Phe-Thr-X-Met-Trp-Tyr-Val-
wherein X indicates any amino acid sequence residue or pharmaceutically acceptable salt thereof and A represents Ser, Asp or Glu, and preferably Ser.

17. A method for isolating a nucleic acid molecule encoding an inhibitor of the extrinsic clotting pathway from triatomine bugs, the method comprising probing a nucleic acid library with a probe as set out in claim 16.

18. An antibody which is capable of binding to an inhibitor of the extrinsic clotting pathway as set out in any one of claims 1 to 6.

19. A method of producing an antibody which is capable of binding to the polypeptide inhibitor of any one of claims 1 to 6, the method comprising using the polypeptide inhibitor as an immunogen.


SEQUENCE LISTING (2 sequences)

SEQ ID NO: 1 - protein, 21 aa, Homo sapiens
DSEEDEEHTIITDTELPPLKL

SEQ ID NO: 2 - protein, 27 aa, Dipetalogaster maximus
  SITE (1)..(1): Xaa represents Ser, Asp or Glu
XMVTNXNMPNPMTGFEKSXFFTXMWYV